(19) United States
(12) Patent Application Publication
(10) Pub. No.: US 2024/0104800 A1
(43) Pub. Date: Mar. 28, 2024

(54) ULTRASOUND IMAGING METHOD AND ULTRASOUND IMAGING APPARATUS

(71) Applicant: SHENZHEN MINDRAY BIO-MEDICAL ELECTRONICS CO., LTD., Shenzhen (CN)

(72) Inventors: Jing LIU, SHENZHEN (CN); Chongchong GUO, SHENZHEN (CN); Bo YANG, SHENZHEN (CN)

(21) Appl. No.: 18/373,909

(22) Filed: Sep. 27, 2023

(30) Foreign Application Priority Data
Sep. 27, 2022 (CN) 202211182508.9

(51) Int. Cl. G06T 11/00

(52) U.S. Cl. CPC G06T 11/006

(57) ABSTRACT

An ultrasound imaging method includes: transmitting a first ultrasound wave to a target tissue via an ultrasound probe; receiving, via the ultrasound probe, echo of the first ultrasound wave that is reflected by the target tissue to obtain an echo channel signal, where the echo channel signal includes a plurality of channel data corresponding to a plurality of receiving array elements of the ultrasound probe; determining a target transformation matrix from a transformation matrix set according to a transmission parameter of the first ultrasound wave and/or a type of the ultrasound probe; transforming the plurality of channel data according to the target transformation matrix to obtain synthetic radio frequency data; performing image processing on the synthetic radio frequency data to obtain image data of the target tissue; and obtaining an image of the target tissue according to the image data, and displaying the image of the target tissue.

### CROSS-REFERENCE TO RELATED APPLICATIONS

This application is based on and claims priority to and benefits of Chinese Patent Application No. 202211182508.9 filed on Sep. 27, 2022. The entire content of the above-referenced application is incorporated herein by reference.

### TECHNICAL FIELD

The disclosure relates to ultrasound imaging, and in particular, to an ultrasound imaging method and an ultrasound imaging apparatus.

### BACKGROUND

Medical diagnostic ultrasound imaging devices obtain ultrasound image information of human tissues and organ structures by means of the propagation of ultrasound waves in a human body. Diagnostic ultrasound plays an extremely important role in modern diagnostic technology due to its advantages of safety, wide adaptability, intuitiveness, availability of repeated examination, strong ability to differentiate soft tissues, strong flexibility, low cost, etc.

In diagnostic ultrasound imaging, the process of obtaining an ultrasound image includes transmitting an ultrasound wave, receiving echoes of the ultrasound wave that are reflected by a human tissue, performing beamforming on the echoes of the ultrasound wave to obtain an echo channel signal of the ultrasound wave, and performing image processing on the echo channel signal of the ultrasound wave to obtain the ultrasound image. An existing beamforming method is mainly a delay-and-sum beamforming method, in which echo channel signals of an ultrasound wave in a plurality of channels are weighted, to obtain a beamformed echo channel signal of the ultrasound wave. Since there are different requirements for transmission, reception, and imaging of an ultrasound wave in different scenarios, a beamforming algorithm needs to be adjusted according to different scenarios. For example, a delay coefficient and an apodization coefficient in the beamforming algorithm need to be adjusted according to a type, a transmission parameter, and an imaging mode of an ultrasound probe. The setting and debugging process is very complicated, which leads to a high cost of usage and development of diagnostic ultrasound imaging, as well as inconvenience to subsequent maintenance.

### SUMMARY

The following is a summary of the subject matter detailed herein. This summary is not to limit the scope of protection of the claims.

Embodiments of the disclosure provide an ultrasound imaging method and an ultrasound imaging device, by which synthetic radio frequency data can be obtained by directly calculating a plurality of channel data received by a plurality of receiving array elements of an ultrasound probe, without needing to adjust parameters in a beamforming algorithm, such that the cost of development is low and maintenance is facilitated.

In an embodiment, an ultrasound imaging method is provided. The method includes:
transmitting a first ultrasound wave to a target tissue via an ultrasound probe;
receiving, via the ultrasound probe, echo of the first ultrasound wave that is reflected by the target tissue to obtain an echo channel signal, where the echo channel signal comprises a plurality of channel data corresponding to a plurality of receiving array elements of the ultrasound probe;
determining a target transformation matrix from a preset transformation matrix set according to a transmission parameter of the first ultrasound wave and/or a type of the ultrasound probe;
transforming the plurality of channel data according to the target transformation matrix to obtain synthetic radio frequency data;
performing image processing on the synthetic radio frequency data to obtain image data of the target tissue; and
obtaining an image of the target tissue according to the image data, and displaying the image of the target tissue.

In an embodiment, an ultrasound imaging apparatus is provided. The apparatus includes:
an ultrasound probe;
a transmitting/receiving circuit configured to control the ultrasound probe to transmit an ultrasound wave to a target tissue and receive ultrasound echoes, to obtain an ultrasound echo signal;
a processor configured to process the ultrasound echo signal to obtain an image of the target tissue; and
a display configured to display the image of the target tissue;
the processor being further configured to perform the ultrasound imaging method in any one of the embodiments of the present disclosure.

In an embodiment, an ultrasound imaging apparatus is provided. The apparatus includes:
an ultrasound probe configured to transmit a first ultrasound wave to a target tissue and receive echoes of the first ultrasound wave, the ultrasound probe comprising a plurality of receiving array elements, where the plurality of receiving array elements are configured to receive the echoes of the first ultrasound wave that are reflected by the target tissue to obtain channel data respectively corresponding to the receiving array elements;
a memory storing a preset transformation matrix set, where the preset transformation matrix set comprises at least one preset transformation matrix;
a processor configured to determine a target transformation matrix from the preset transformation matrix set according to a transmission parameter of the first ultrasound wave and/or a type of the ultrasound probe; and
a synthetic processor configured to transform the plurality of channel data according to the target transformation matrix to obtain synthetic radio frequency data;
where the processor being further configured to perform image processing on the synthetic radio frequency data to obtain image data of the target tissue, to obtain an image of the target tissue according to the image data, and to display the image of the target tissue.

In an embodiment, an electronic device is provided, which includes a memory and a processor, where the memory stores a computer program, and when the processor executes the computer program, the ultrasound imaging method in any one of the embodiments of the present disclosure is implemented.

In an embodiment, a computer storage medium is provided, which has a computer program stored thereon and is applied to an ultrasound imaging device, where when the computer program is executed by a processor, the ultrasound imaging method in any one of the embodiments of the present disclosure is implemented.

In an embodiment, a computer program product or computer program is provided, which includes computer instructions, where the computer instructions are stored in a computer-readable storage medium. A processor of a computer device reads the computer instructions from the computer-readable storage medium, and the processor executes the computer instructions, so that the computer device performs the ultrasound imaging method in any one of the embodiments of the present disclosure.

In the embodiments of the disclosure, a target transformation matrix is determined from a preset transformation matrix set according to a transmission parameter of a first ultrasound wave and/or a type of an ultrasound probe; then, a plurality of channel data received by a plurality of receiving array elements of the ultrasound probe are transformed according to the target transformation matrix, to obtain synthetic radio frequency data; image processing is performed on the synthetic radio frequency data to obtain image data of a target tissue; and an image of the target tissue is obtained according to the image data, and the image of the target tissue is displayed. In the embodiments of the disclosure, the synthetic radio frequency data is obtained by directly calculating the plurality of channel data received by the plurality of receiving array elements of the ultrasound probe, without needing to adjust parameters in a beamforming algorithm. Therefore, a complex setting and debugging process is avoided, the cost of development of ultrasound imaging can be effectively reduced, and subsequent maintenance is facilitated.

### BRIEF DESCRIPTION OF THE DRAWINGS

The accompanying drawings are used for providing a further understanding of the technical solution in the disclosure and constitute a part of this specification, and are used, together with the embodiments of the disclosure, for explaining the technical solution in the disclosure but do not limit the technical solution in the disclosure.

FIG. 1 is a block diagram of a structure of an ultrasound imaging apparatus according to an embodiment of the disclosure;

FIG. 2 is a flowchart of an ultrasound imaging method according to an embodiment of the disclosure;

FIG. 3 is a schematic block diagram of signal processing of an ultrasound imaging apparatus according to an embodiment of the disclosure;

FIG. 4 is a flowchart of obtaining a preset transformation matrix according to an embodiment of the disclosure;

FIG. 5 is a schematic diagram of a structure of an ultrasound detection simulation model according to an embodiment of the disclosure;

FIG. 6 is a flowchart of step 420 in FIG. 4;

FIG. 7 is a schematic diagram of channel data of a channel data matrix;

FIG. 8 is a flowchart of step 620 in FIG. 6;

FIG. 9 is a schematic diagram of an experimental environment according to an embodiment of the disclosure;

FIG. 10 is a flowchart of obtaining a preset transformation matrix according to an embodiment of the disclosure;

FIG. 11 is a flowchart of step 1030 in FIG. 10;

FIG. 12 is a flowchart of step 1120 in FIG. 11; and

FIG. 13 is a block diagram of a structure of an ultrasound imaging apparatus according to an embodiment.

### DETAILED DESCRIPTION

The disclosure will be further described below with reference to the accompanying drawings and embodiments. The embodiments described should not be construed as limiting the disclosure. All other embodiments obtained by those ordinary skill in the art without creative efforts shall fall within the scope of protection of the disclosure.

In the following description, “some embodiments” are involved, which describes a subset of all possible embodiments. However, it may be understood that “some embodiments” may be the same subset or different subsets of all possible embodiments, and may be combined with each other in the case of no conflict.

Unless otherwise defined, all technical and scientific terms used herein have the same meanings as those commonly understood by those skilled in the art to which the disclosure belongs. The terms used herein are merely for the purpose of describing embodiments of the disclosure, and are not to limit the disclosure.

Medical diagnostic ultrasound imaging devices obtain ultrasound image information of human tissues and organ structures by means of the propagation of ultrasound waves in a human body. Diagnostic ultrasound plays an extremely important role in modern diagnostic technology due to its advantages of safety, wide adaptability, intuitiveness, availability of repeated examination, strong ability to differentiate soft tissues, strong flexibility, low cost, etc.

In diagnostic ultrasound imaging, the process of obtaining an ultrasound image includes transmitting an ultrasound wave, receiving echoes of the ultrasound wave that are reflected by a human tissue, performing beamforming on the echoes of the ultrasound wave to obtain an echo channel signal of the ultrasound wave, and performing image processing on the echo channel signal of the ultrasound wave to obtain the ultrasound image. An existing beamforming method is mainly a delay-and-sum beamforming method, in which echo channel signals of an ultrasound wave in a plurality of channels are weighted, to obtain a beamformed echo channel signal of the ultrasound wave. Since there are different requirements for transmission, reception, and imaging of an ultrasound wave in different scenarios, a beamforming algorithm needs to be adjusted according to different scenarios. For example, a delay coefficient and an apodization coefficient in the beamforming algorithm need to be adjusted according to a type (such as a linear array probe, a convex array probe, and a phased array probe), a transmission parameter (such as a diverging wave transmission parameter, a focused wave transmission parameter, and a plane wave transmission parameter), and an imaging mode (such as a B image mode and a C image mode) of an ultrasound probe. The setting and debugging process is very complicated, which leads to a high cost of usage and development of diagnostic ultrasound imaging, as well as inconvenience to subsequent maintenance.

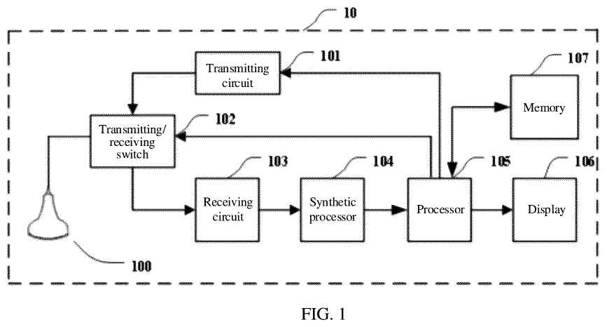

In order to solve the above problems, embodiments of the disclosure provide an ultrasound imaging method and an ultrasound imaging apparatus. FIG. 1 is a schematic block diagram of a structure of an ultrasound imaging apparatus in an embodiment of the disclosure. The ultrasound imaging apparatus 10 may include an ultrasound probe 100, a transmitting circuit 101, a transmitting/receiving switch 102, a receiving circuit 103, a synthetic processor 104, a processor 105, a display 106, and a memory 107.

The ultrasound probe 100 includes a transducer (not shown) composed of a plurality of array elements arranged in an array. The plurality of array elements is arranged into a row to form a linear array or into a two-dimensional matrix to form an area array. In an embodiment, the plurality of array elements may form a convex array. The array element is configured to transmit an ultrasound beam according to an excitation electrical signal, or transform a received ultrasound beam into an electrical signal. An array element for transmitting an ultrasound wave is a transmitting array element, and an array element for receiving an ultrasound wave is a receiving array element. The transmitting array element and the receiving array element may be multiplexed, that is, one array element may be responsible for both transmission and receiving. Therefore, each array element may be configured to implement mutual conversion of an electric pulse signal and an ultrasound beam, so as to transmit an ultrasound wave to a target region of a human tissue, or may be configured to receive echoes of the ultrasound wave that are reflected by the tissue. During ultrasound detection, the transmitting/receiving switch 102 may be used to control which array elements are used to transmit an ultrasound beam and which array elements are used to receive an ultrasound beam, or control the array elements to be used to transmit an ultrasound beam or receive echoes of the ultrasound beam in different slots. The array elements participating in transmission of the ultrasound wave can be simultaneously excited by the electrical signal, so as to simultaneously transmit the ultrasound wave; or the array elements participating in transmission of the ultrasound wave may be excited by several electrical signals having a time interval, so as to continuously transmit ultrasound waves having a time interval. In an embodiment, a plurality of array elements may together form transmitting array elements or receiving array elements.

The transmitting circuit 101 is configured to generate a transmitting sequence under control of the processor 105. The transmitting sequence is used to control some or all of the plurality of array elements to transmit an ultrasound wave to a biological tissue. Parameters of the transmitting sequence include positions of the transmitting array elements, the number of the array elements, and transmission parameters of the ultrasound beam (such as amplitude, frequency, times of transmission, transmission interval, transmission angle, waveform, and focusing position). In some cases, the transmitting circuit 101 is further configured to delay a phase of the transmitted beam, such that different transmitting array elements transmit ultrasound waves at different moments, and ultrasound beams transmitted can thus be focused in a predetermined region of interest. The parameters of the transmitting sequence vary depending on working modes, such as B image mode, C image mode, and D image mode (Doppler mode).

The receiving circuit 103 is configured to receive the electrical signal of the ultrasound echo from the ultrasound probe 100, and process the electrical signal of the ultrasound echo. Each receiving array element of the ultrasound probe 100 receives an echo of the ultrasound wave that is reflected by the target tissue, and obtains channel data received by each array element. In other words, each receiving array element in the ultrasound probe 100 that is responsible for receiving echoes of the ultrasound wave receives the echoes of the ultrasound wave, and a piece of channel data is then formed, that is, the electrical signal of the echoes of the ultrasound wave includes a plurality of channel data. The receiving circuit 103 processes a plurality of channels of data of the electrical signal. The receiving circuit 103 may include one or more amplifiers, analog-to-digital converters (ADCs), etc. The amplifier is configured to amplify the received electrical signal of the ultrasound echo after proper gain compensation. The analog-to-digital converter is configured to sample the analog echo signal at predetermined time intervals, so as to convert same into a digital signal. The digital echo signal still retains amplitude information, frequency information, and phase information. Data output by the receiving circuit 103 may be output to the synthetic processor 104 for processing, or output to the memory 107 for storage.

The synthetic processor 104 is connected to the receiving circuit 103 using a signal, and is configured to perform synthetic processing on each piece of channel data in a signal output by the receiving circuit 103. Because an ultrasound wave receiving point in a detected tissue has different distances from receiving array elements, channel data of the same receiving point that is output by different receiving array elements have a delay difference, which requires each piece of channel data to be transformed, and phases to be aligned, to obtain synthetic radio frequency data. The synthetic radio frequency data output by the synthetic processor 104 is also referred to as radio frequency data (RF data). In some embodiments, the synthetic processor 104 may output the radio frequency data to the memory 107 for caching or storage, or directly output the radio frequency data to an image processing module of the processor 105 for image processing.

The synthetic processor 104 may perform the above functions in hardware, firmware, or software. For example, the synthetic processor 104 may include a central controller circuit (CPU), one or more microprocessors, or any other electronic component capable of processing input data according to logical instructions. In an embodiment, the synthetic processor includes a video memory and a graphics processing unit (GPU), the video memory being configured to cache a target transformation matrix and a plurality of channel data, and the GPU being configured to obtain the target transformation matrix and the plurality of channel data from the video memory, and transform the plurality of channel data according to the target transformation matrix, to obtain the synthetic radio frequency data. The video memory and the GPU may also be replaced by other circuits or devices having data storage and computing capabilities, which depends on the computational complexity of solving an inverse problem. In an embodiment, the synthetic processor 104 may use an application-specific integrated circuit (ASIC), a field-programmable gate array (FPGA), etc., or may use a combination of two or more devices. When implemented in software, the synthetic processor 104 may execute instructions stored on a tangible and non-transitory computer-readable medium (e.g., the memory 107), to perform beamforming calculation using any suitable beamforming method.

The memory 107 stores a preset transformation matrix set. The preset transformation matrix set includes at least one preset transformation matrix, and the preset transformation matrix is used by the synthetic processor 104 to transform the plurality of channel data. The memory 107 may be a tangible and non-transitory computer-readable medium, for example, may be a flash memory card, a solid-state memory, a hard disk, etc., for storing data or a program. In an embodiment, the memory 107 may also be configured to store the acquired ultrasound data or an image frame that is generated by the processor 105 but is not immediately displayed; or the memory 107 may store a graphical user interface, one or more default image display settings, and programming instructions for the processor, a beamforming circuit, or an IQ demodulation module.

The processor 105 is configured to be a central controller circuit (CPU), one or more microprocessors, a graphics controller circuit (GPU), or any other electronic component capable of processing input data according to logical instructions. The processor may control peripheral electronic components or read and/or store data from and/or to the memory 107 according to input instructions or predetermined instructions, or may process input data by executing a program in the memory 107. For example, one or more processing operations are performed on acquired ultrasound data in one or more working modes, where the processor 105 can read elements from the preset transformation matrix set in the memory 107, and the processor 105 may determine a corresponding target transformation matrix from the preset transformation matrix set according to the transmission parameter of the ultrasound wave and/or a type of the ultrasound probe. In an embodiment, the processor 105 sends the target transformation matrix to the synthetic processor 104, so that the synthetic processor transforms the plurality of channel data according to the target transformation matrix, to obtain the synthetic radio frequency data. In another embodiment, the processor 105 may also be the synthetic processor 104 (or integrated with the synthetic processor 104). In other words, the processor 105 transforms the plurality of channel data according to the determined target transformation matrix, to obtain the synthetic radio frequency data.

In an embodiment, the processing operations further include adjusting or defining the form of an ultrasound wave transmitted by the ultrasound probe 100 and generating various image frames for subsequent display on the display 106 of a human-machine interaction apparatus, or adjusting or defining the content and form displayed on the display 106, or adjusting one or more image display settings (e.g., ultrasound image, interface component, locating of a region of interest) displayed on the display 106.

The image processing module of the processor 105 is configured to perform image processing on the synthetic radio frequency data output by the synthetic processor 104, to obtain image data of the target tissue, for example, to generate a grayscale image for signal strength variations within a scanning range. The grayscale image reflects an internal anatomical structure of the tissue, and is referred to as a B-mode image. The image processing module may output the B-mode image to the display 106 of the human-machine interaction apparatus for display. The human-machine interaction apparatus is configured to perform human-machine interaction, that is, to receive the user's input and output visual information. The human-machine interaction apparatus may receive the user's input using a keyboard, an operation button, a mouse, a trackball, etc., or using a touchscreen integrated with the display. The human-machine apparatus outputs the visual information using the display 106.

It should be noted that the structure in FIG. 1 is only for illustration, and more or fewer components than those shown in FIG. 1 may be included, or a configuration different from that shown in FIG. 1 may be used. The components shown in FIG. 1 may be implemented in hardware and/or software.

In an embodiment of the disclosure, the display 106 of the foregoing ultrasound imaging apparatus 10 may be a touch display screen, a liquid crystal display screen, etc., or may be an independent display device, such as a liquid crystal display or a television, independent of the ultrasound imaging device 10, or may be a display screen on an electronic device, such as a mobile phone and a tablet computer.

In an embodiment of the disclosure, the memory 107 of the foregoing ultrasound imaging apparatus 10 may be a flash memory card, a solid state memory, a hard disk, etc.

In an embodiment of the disclosure, a computer-readable storage medium is further provided. The computer-readable storage medium stores a plurality of program instructions. After the plurality of program instructions are invoked and executed by the processor 105, some or all of the steps or any combination of the steps in the method for processing data of an ultrasound wave in the embodiments of the disclosure may be performed.

In an embodiment, the computer-readable storage medium may be the memory 107, which may be a non-volatile storage medium, such as a flash memory card, a solid-state memory, and a hard disk.

In an embodiment of the disclosure, the processor 105 of the foregoing ultrasound imaging apparatus 10 may be implemented in software, hardware, firmware, or a combination thereof, and may use circuits, one or more application-specific integrated circuits (ASICs), one or more general integrated circuits, one or more microprocessors, one or more programmable logic devices, or a combination of the foregoing circuits or devices, or other suitable circuits or devices, such that the processor 105 can perform the corresponding steps of the method for processing data of an ultrasound wave in the embodiments of the disclosure.

The ultrasound imaging method of the embodiments of the disclosure are described below in conjunction with the accompanying drawings.

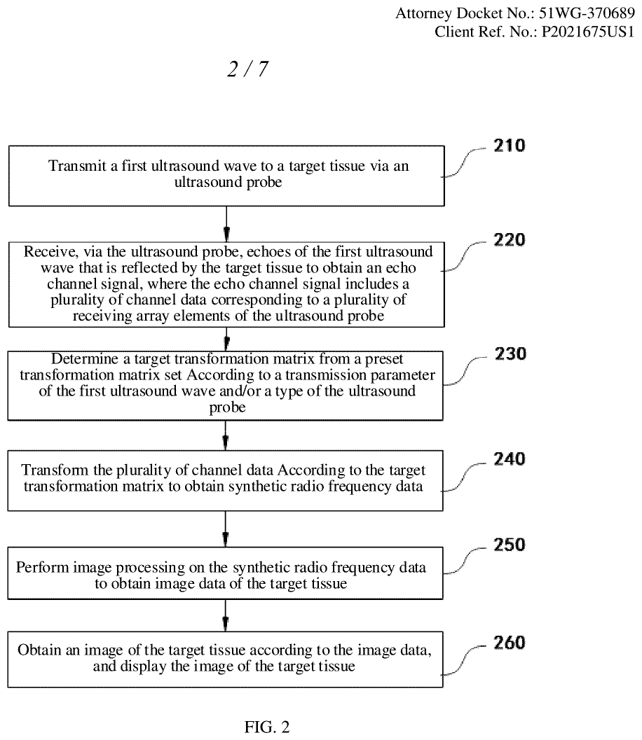

In conjunction with the schematic block diagram of a structure of the ultrasound imaging apparatus 10 shown in FIG. 1, referring to FIG. 2, the ultrasound imaging method provided in an embodiment of the disclosure may include steps 210 to 260 as follows.

In step 210, an ultrasound probe transmits a first ultrasound wave to a target tissue.

In this step, the ultrasound imaging apparatus 10 transmits, by the ultrasound probe 100, the first ultrasound wave to the target tissue. The target tissue may be tissues at different parts of a human body, such as heart, liver, and ovaries. The processor 105 controls the transmitting circuit 101 to generate a transmitting sequence of the ultrasound wave according to a preset or user-set transmission parameter. The transmitting sequence is used to control some or all of the plurality of array elements to transmit an ultrasound wave to a biological tissue. Parameters of the transmitting sequence include positions of the transmitting array elements, the number of the array elements, and transmission parameters of the ultrasound beam (such as amplitude, frequency, times of transmission, transmission interval, transmission angle, waveform, and focusing position). The ultrasound probe 100 can transmit the ultrasound wave a plurality of times according to the transmitting sequence of the ultrasound wave. In some cases, the transmitting circuit 101 is further configured to delay a phase of the transmitted beam, such that different transmitting array elements transmit ultrasound waves at different moments, and ultrasound beams transmitted can thus be focused in a predetermined region of interest, to transmit a focused wave, a plane wave, or a diverging wave. Therefore, the respective transmission parameters of the ultrasound beam are a focused wave transmission parameter, a plane wave transmission parameter, and a diverging wave transmission parameter. In addition, for different probes, such as a linear array probe, a convex array probe, and a phased array probe, their respective transmission parameters also vary, which can then be classified into a linear array probe transmission parameter, a convex array probe transmission parameter, and a phased array probe transmission parameter. In addition, there are different working modes for the ultrasound imaging apparatus 10 according to the need of detection, and a transmission parameter also needs to be set under different working modes. For example, for a B image mode (reflecting the anatomical structure of the tissue), a C image mode (reflecting the anatomical structure of the tissue and blood flow information), a D image mode (a Doppler mode), an E image mode (an ultrasound elasticity image mode), or an M image mode (also referred to as an ultrasound echocardiogram), the respective B image mode transmission parameter, C image mode transmission parameter, D image mode transmission parameter, or E image mode transmission parameter is set.

In step 220, the ultrasound probe receives an echo channel signal of the first ultrasound wave that is reflected by the target tissue, where the echo channel signal includes a plurality of channel data corresponding to a plurality of receiving array elements of the ultrasound probe.

In this step, after receiving the first ultrasound wave, the target tissue reflects the echoes of the first ultrasound wave, such that the echoes of the first ultrasound wave that are reflected by the target tissue are received by the ultrasound probe 100, and then sent to the receiving circuit 103 to be converted into an electrical signal. The receiving array elements of the ultrasound probe 100 may each receive an ultrasound signal, and respectively generate a corresponding electrical signal. These electrical signals are processed by the receiving circuit 103 to form an echo channel signal, where the echo channel signal includes the plurality of channel data corresponding to the plurality of receiving array elements of the ultrasound probe, that is, each receiving array element corresponds to one piece of channel data. In an embodiment, all array elements of the ultrasound probe 100 are each used as receiving array elements to receive an echo signal of the first ultrasound wave. In another embodiment, only some of the array elements of the ultrasound probe 100 are each used as receiving array elements. In another embodiment, a plurality of array elements of the ultrasound probe 100 are combined to form one receiving array element. In these cases above, it is also the case where one receiving array element corresponds to one piece of channel data.

In step 230, a target transformation matrix is determined from a preset transformation matrix set according to a transmission parameter of the first ultrasound wave and/or a type of the ultrasound probe.

In this step, the processor 105 determines the target transformation matrix from the preset transformation matrix set in the memory 107 according to the transmission parameter and/or the type of the ultrasound probe in step 210. The preset transformation matrix set stores a plurality of pre-calculated preset transfer matrices. Different preset transfer matrices correspond to different transmission environments, for example, to different transmission parameters and/or different types of the ultrasound probe. In an embodiment, each preset transformation matrix in the preset transformation matrix set is recorded and stored during experiments or simulation by simulating a propagation environment of an ultrasound wave, and there is a correspondence between a transmission parameter used during the experiments or simulation and the preset transformation matrix recorded during the experiments or simulation, where the preset transformation matrix may also be referred to as a measurement matrix.

In an embodiment, a mapping relationship between the transmission parameter and/or the type of the ultrasound probe and each preset transformation matrix can be pre-constructed, and a target transformation matrix corresponding to the current transmission parameter is searched for through the mapping relationship. For example, different transmission parameters may be numbered or named, and then, according to the number or name of the transmission parameter/the type of the ultrasound probe type used in step 210, a corresponding preset transformation matrix is searched for through the mapping relationship. The preset transformation matrix is used as the target transformation matrix. In an embodiment, the preset transfer matrices in the preset transformation matrix set are sparse matrices, whereby the storage space of the memory 107 can be saved.

In another embodiment, the target transformation matrix may also be determined from the preset transformation matrix set by setting a condition. For example, a corresponding preset transformation matrix may be determined by making a comparative determination on an attribute of a transmission parameter and a preset condition. The attribute of the transmission parameter may be one of or a combination of transmission amplitude, transmission frequency, times of transmission, transmission interval, transmission angle, waveform, focusing position, and transmission type (a focused wave, a plane wave, or a diverging wave) of the ultrasound wave. The set condition may be an exact value or a threshold range. In this way, the attribute of the transmission parameter is compared with the preset condition to determine the corresponding target transformation matrix. In an embodiment, the transmission parameter and the type of the ultrasound probe can be jointly used as a condition determination factor. In another embodiment, a preset transformation matrix corresponding to the current transmission parameter and/or the ultrasound probe may be determined from the preset transformation matrix set, and then, further operations are performed on the preset transformation matrix, to obtain the target transformation matrix, for example, mathematical calculation is performed on the preset transformation matrix and the transmission parameter, to obtain the target transformation matrix. The preset matrix in the preset transformation matrix set may be a master to be further processed, which is subjected to mathematical calculation together with the transmission parameter, to obtain a more accurate target transformation matrix that meets the current transmission condition.

In step 240, the plurality of channel data is transformed according to the target transformation matrix to obtain synthetic radio frequency data.

In this step, each receiving array element in the ultrasound probe 100 corresponds to one piece of channel data. Therefore, the echo channel signal is a data matrix, and elements of the data matrix are channel data. Thus, mathematical transformation operations can be performed on the echo channel signal and the target transformation matrix, to obtain a transformed data matrix. The transformed data matrix is the synthetic radio frequency data. In an embodiment, the synthetic radio frequency data can be obtained by directly performing mathematical operations, such as matrix multiplication, or transpose of the target transformation matrix and then matrix multiplication, on the echo channel signal and the target transformation matrix. In another embodiment, the echo channel signal and the target transformation matrix cannot be directly multiplied, or there is no solution after direct multiplication. However, since the preset transformation matrix has a linear forward relationship with the corresponding transmission parameter when the preset transformation matrix is recorded in an experimental or simulation stage, the plurality of channel data in the echo channel signal, the target transformation matrix, and the synthetic radio frequency data constitute a linear forward model, such that an inverse problem for the linear forward model can be solved according to the target transformation matrix and the plurality of channel data in the echo channel signal, to obtain the synthetic radio frequency data.

In an embodiment, the plurality of channel data is rearranged into column vectors according to a channel order of the receiving array elements of the ultrasound probe, to obtain the plurality of channel data in a column vector form. The plurality of channel data in the column vector form are transformed according to the target transformation matrix, to obtain the synthetic radio frequency data in a column vector form. Rearranging the channel data in the echo channel signal into column vectors, to obtain the plurality of channel data in a column vector form can facilitate matrix operations and transformation processing, thereby reducing the amount of calculation.

As described above, a predicted transformation matrix is recorded and calculated in advance according to experiments or simulation. Therefore, the linear forward model can be expressed as:

Y=Σᵢ₌₁^{N}XᵢTᵢ,

where Y represents the echo channel signal received by the ultrasound probe 100, Xᵢ represents scattering intensity of the i^{th} beamforming point, and Tᵢ represents data in the i^{th} column of the predicted transformation matrix, i being a natural number. Therefore, each beamforming point can be measured during the experiments or simulation, to obtain transformation matrix data in each column, and then obtain the predicted transformation matrix T. Therefore, the above linear forward model can be further expressed as:

Y=TX,

where Y is the echo channel signal, X is the synthetic radio frequency data, T is the target transformation matrix determined from the preset transformation matrix set according to the transmission parameter and/or the type of the ultrasound probe, and the plurality of channel data are rearranged into column vectors according to the channel order of the receiving array elements of the ultrasound probe, to obtain the echo channel signal Y, both Y and X being data in a column vector form.

In an embodiment, a number Ne of receiving array elements are provided on the ultrasound probe 100, each receiving array element correspondingly receiving one piece of channel data. Since ultrasound detection requires the detection of a target tissue with a certain depth, each transmitting array element transmits, to the target tissue, the first ultrasound wave with a number Ns of preset or user-set sampling points. Each sampling point corresponds to the detection depth of each ultrasound wave, and the density of the sampling points affects the resolution of the image. Under control of the transmission parameter, the transmitting circuit 101 generates a transmitting sequence of the ultrasound wave to excite the transmitting array elements to transmit the ultrasound wave a number Nt of times, that is, to reflect the ultrasound wave a number Nt of times, where Ne, Ns, and Nt are all natural numbers. Therefore, the ultrasound probe 100 can receive a number Ns*Ne*Nt of channel data, and these channel data are rearranged into column vectors to obtain the echo channel signal Y. In an embodiment, the receiving array elements of the ultrasound probe 100 obtain a number Ns*Ne of pieces of matrix data, the pieces of matrix data are vectorized to obtain a column vector Yᵢ, and the Nt times of ultrasound transmission are then correspondingly arranged into the same column of vectors. In this way, Y is a column vector with a number Ns*Ne*Nt of elements, where each element is channel data generated by the receiving array element receiving the echo of the ultrasound wave.

Since the echo channel signal Y and the target transformation matrix T are known, an inverse problem for the linear forward model Y=TX can be solved, to obtain the synthetic radio frequency data X.

In an embodiment, the inverse problem can be solved by means of numerical solving, which involves performing matrix inversion on the target transformation matrix T, to obtain a target inverse transformation matrix T^{H}, and performing matrix multiplication on the target inverse transformation matrix T^{H} and the echo channel signal Y, i.e., X=YT^{H}, to obtain the synthetic radio frequency data. This method requires only one matrix multiplication operation, making calculation simple.

In another embodiment, least-squares transformation may be performed to solve for an inverse matrix, to obtain the synthetic radio frequency data. That is, X=(T^{H}T)⁻¹T^{H}Y. The least-squares solving requires solving for an inverse matrix of TT^{H}. When TT^{H} is ill-posed, there is no inverse matrix for the matrix. To solve this problem, solving can be performed using a related regularization method, such as Tikhonov regularization. In this case, X=(T^{H}T+rT)⁻¹T^{H}Y, where r is a regular coefficient, and r is a constant.

In another embodiment, solving can be performed by means of compressed sensing for the target transformation matrix T and the echo channel signal Y, to obtain the synthetic radio frequency data X. In an embodiment, the target transformation matrix T is a sparse matrix, and because it is assumed in compressed sensing that the unknown vector X to be sought is sparse, this assumption can be applied to solve for the synthetic radio frequency data X.

In another embodiment, the target transformation matrix T and the echo channel signal Y can be input into a pre-trained deep learning model for inverse problem solving for processing, to obtain the synthetic radio frequency data X.

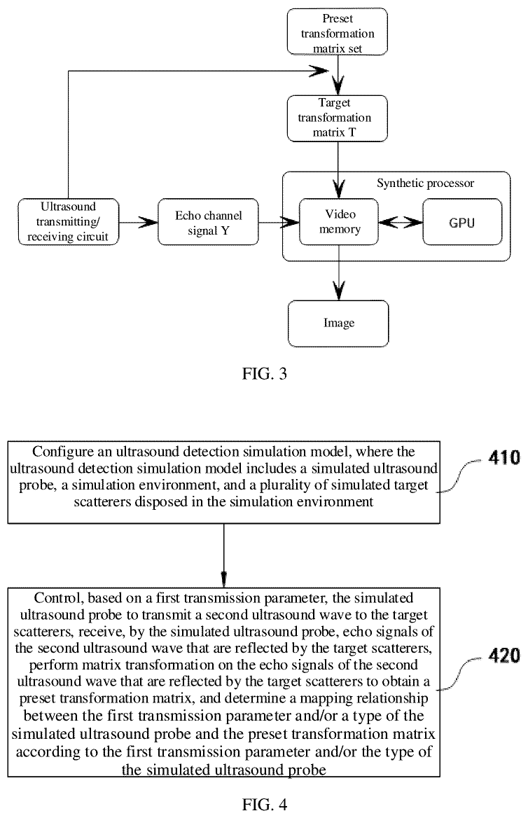

Referring to FIG. 3, in an embodiment, the synthetic processor 104 performs this step 240. For example, the synthetic processor 104 obtains the echo channel signal Y output by the receiving circuit 103, and obtains the target transformation matrix T determined in step 230 above, to solve for the synthetic radio frequency data X in the linear forward model. In an embodiment, the synthetic processor 104 includes a video memory and a graphics processing unit (GPU), the video memory being configured to cache the target transformation matrix T and the echo channel signal Y, and the GPU being configured to obtain the target transformation matrix T and the echo channel signal Y from the video memory, and transform the plurality of channel data in the echo channel signal Y according to the target transformation matrix T, to obtain the synthetic radio frequency data. Due to powerful graphics computing performance, the GPU can speed up the operation of the inverse problem, and improve the calculation speed of solving for the synthetic radio frequency data X. By separating the GPU from the processor 105 to effectively share the computing load of the processor 105, the computing requirements of the processor 105 may be lowered, and costs are further reduced.

In another embodiment, the synthetic processor 104 may also be a computing module within the processor 105, for example, the synthetic processor 104 is an integrated display unit integrated into the processor 105, such that the integrated display unit and the CPU share the same memory. In another embodiment, the processor 105 may also be used to complete the operation of solving for the synthetic radio frequency data X in this step 240.

In step 250, image processing is performed on the synthetic radio frequency data, to obtain image data of the target tissue.

In this step, an image processing module of the processor 05 is used to perform image processing on the synthetic radio frequency data X output by the synthetic processor 104, to obtain the image data of the target tissue.

In step 260, an image of the target tissue is obtained according to the image data, and the image of the target tissue is displayed.

In this step, the image of the target tissue within a scanning range is obtained according to the image data in step 250 above. In an embodiment, the image of the target tissue is a grayscale image for signal strength variations. The grayscale image reflects a tissue structure of the target tissue. In another embodiment, the tissue image may be a B-mode image or an E-mode image that reflects only the internal anatomical structure of the tissue, or a C-mode image or an M-mode image that reflects the internal anatomical structure of the tissue and blood flow information. In this step, the display 106 is used to display the tissue image of the target tissue.

In the ultrasound imaging apparatus and the ultrasound imaging method provided in the above embodiments, a target transformation matrix is determined from a preset transformation matrix set according to a transmission parameter of a first ultrasound wave and/or a type of an ultrasound probe; then, a plurality of channel data received by a plurality of receiving array elements of the ultrasound probe are transformed according to the target transformation matrix, to obtain synthetic radio frequency data; image processing is performed on the synthetic radio frequency data to obtain image data of a target tissue; and an image of the target tissue is obtained according to the image data, and the image of the target tissue is displayed. In the embodiments of the disclosure, the synthetic radio frequency data is obtained by directly calculating the plurality of channel data received by the plurality of receiving array elements of the ultrasound probe, without needing to adjust parameters in a beamforming algorithm. Therefore, a complex setting and debugging process is avoided, the cost of development of ultrasound imaging can be effectively reduced, and subsequent maintenance is facilitated.

According to the ultrasound imaging apparatus and the ultrasound imaging method provided in some of the above embodiments, the method for solving an inverse problem for the linear forward model is used to replace the existing beamforming method which requires delay-and-sum steps. For example, in the delay-and-sum beamforming method in the prior art, a set of delays may be calculated at beamforming points. If the delays are accurate, the sampling point with the highest amplitude of channel data in each channel may be selected for the set of delays, but delaying and apodization in the prior art need to be calculated according to a collective propagation path of an ultrasound sound field, and the calculation result is inaccurate. Therefore, the beamforming method in the prior art has a low quality and a complicated setting and debugging process. However, in the embodiments of the disclosure, the target transformation matrix comes from channel data Ti corresponding to the beamforming points during experiments and simulation, and Ti has automatically contained amplitude information of each channel signal. When a numerical method for solving X=T^{H}Y is used, if only the sampling point with the highest amplitude in the channel signal is retained when Ti is obtained, and the position is set to 1 while other positions are set to 0, the disclosure is equivalent to delay-and-sum beamforming in this case. The difference lies in that in this case, the delay is calculated without assuming a morphology of the sound field, and is exactly obtained from Ti under a transmission parameter. Therefore, the delay in this case is totally accurate, and a higher beamforming quality can be obtained compared with the prior art. When the transmission parameter is adjusted, Ti will automatically change, and in this case, delay information will also change automatically. Therefore, the disclosure can well adapt to the change in the transmission parameter, greatly increasing the scalability of an ultrasound system. When solving the inverse problem, in addition to the numerical method for solving X=T^{H}Y, a variety of optional solving methods are also provided, which apply different constraints and can obtain different imaging effects compared with the method for X=T^{H}Y. Therefore, under the same architecture, the ultrasound imaging apparatus and the ultrasound imaging method provided in the embodiments of the disclosure can apply a variety of beamforming algorithms, and present strong compatibility. In addition, in the embodiments of the disclosure, since there is no need to make an assumption about the morphology of the sound field, the apodization coefficient no longer needs to be finely adjusted according to an approximate degree of the sound field after the probe or transmission parameter changes, which greatly reduces the workload of personnel for image optimization and the complexity of subsequent maintenance.

The preset transformation matrix set in the memory 107 includes a plurality of preset transfer matrices, and these preset transfer matrices can be obtained by means of simulation or experiments.

Referring to FIG. 4, in an embodiment, the preset transformation matrix is obtained through steps 410 and 420 as follows.

In step 410, an ultrasound detection simulation model is configured, where the ultrasound detection simulation model includes a simulated ultrasound probe, a simulation environment, and a plurality of simulated target scatterers disposed in the simulation environment.

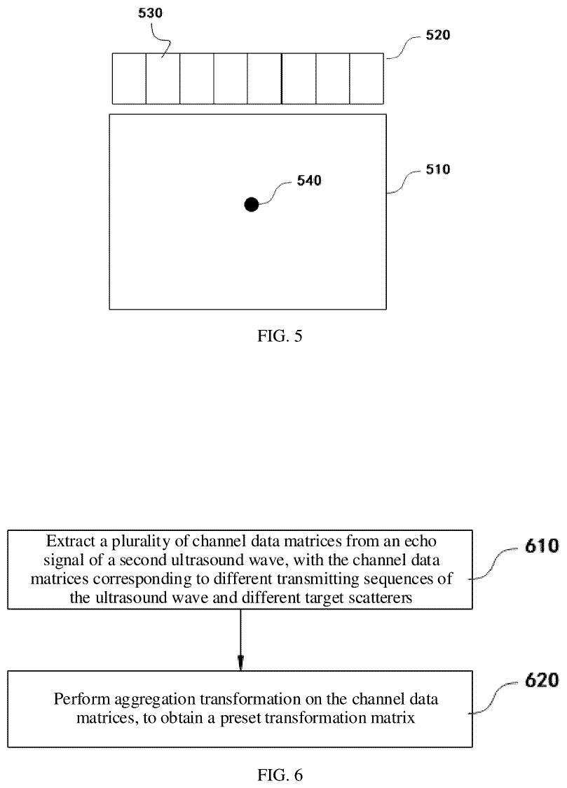

In this step, referring to an embodiment shown in FIG. 5, the configuration of the ultrasound detection simulation model may be simulated using a computer in this step, for example, using ultrasound simulation software. The ultrasound detection simulation model includes a simulation environment 510 for simulating the transmission of an ultrasound wave, and a simulated ultrasound probe 520. The simulated ultrasound probe 520 includes a plurality of array elements 530, and the array elements 530 are used as transmitting array elements in a transmission stage of the ultrasound wave, and are used as receiving array elements in a receiving stage of the ultrasound wave. A target scatterer 540 is disposed in the simulation environment 510. In an embodiment, it is possible to simultaneously place a plurality of target scatterers 540 in the simulation environment 510, and each target scatterer 540 corresponds to a beamforming point. In another embodiment, it is also possible to set one target scatterer 540 and measure same, and then, a plurality of groups of measurement data are formed by continuously adjusting the position of the target scatterer 540. Referring to FIG. 5, the simulated ultrasound probe 520 is a simulated linear array probe. Certainly, a simulated convex array probe or a simulated phased array probe may also be set as needed.

In step 420, the simulated ultrasound probe is controlled, according to a first transmission parameter, to send a second ultrasound wave to the target scatterers, echo signals of the second ultrasound wave that are reflected by the target scatterers are received by the simulated ultrasound probe, matrix transformation is performed on the echo signals of the second ultrasound wave that are reflected by the target scatterers, to obtain the preset transformation matrix, and a mapping relationship with the preset transformation matrix is determined according to the first transmission parameter and/or a type of the simulated ultrasound probe.

In this step, the simulated ultrasound probe 520 sends a second ultrasound wave according to the first transmission parameter. The second ultrasound wave is a simulated ultrasound signal. The second ultrasound wave is simulated for propagation in the simulation environment 510 and is reflected at the positions of the target scatterers, to generate echo signals of the second ultrasound wave that are reflected at the positions of the target scatterers. The echo signals of the second ultrasound wave are received by a plurality of array elements 530 of the simulated ultrasound probe 520, and echo signals are then formed. Matrix transformation is performed on the echo signals of the second ultrasound wave that are reflected by the target scatterers, to obtain the preset transformation matrix, and the preset transformation matrix is added to the preset transformation matrix set. If the current preset transformation matrix is the first matrix, an empty preset transformation matrix set is created, and the current preset transformation matrix is placed into the preset transformation matrix set. In addition, in an embodiment, a relationship between the first transmission parameter and the current preset transformation matrix is also recorded such that the corresponding preset transformation matrix can be found according to the first transmission parameter. In an embodiment, the relationship between the first transmission parameter and the current preset transformation matrix is a one-to-one mapping relationship. In some other embodiments, the relationship between the first transmission parameter and the current preset transformation matrix is a conditional relationship or a mathematical operation relationship. In another embodiment, the relationship between the type of the simulated ultrasound probe 520 and the current preset transformation matrix is recorded. In another embodiment, a relationship between the first transmission parameter, the type of the simulated ultrasound probe 520, and the current preset transformation matrix is recorded.

When the calculation of one preset transformation matrix is completed, the first transmission parameter and/or the type of the simulated ultrasound probe 520 are/is changed for re-measurement, and after a new preset transformation matrix is obtained through calculation, the current preset transformation matrix is added to the preset transformation matrix set, and a relationship between the first transmission parameter and/or the simulated ultrasound probe 520 and the current preset transformation matrix is stored. In an embodiment, the memory 107 is used to store the preset transformation matrix set, and the relationship between the first transmission parameter and/or the simulated ultrasound probe 520 and the current preset transformation matrix.

Referring to FIG. 6, in an embodiment, performing matrix transformation on the echo signals of the second ultrasound wave that are reflected by the target scatterers, to obtain the preset transformation matrix, and determining a mapping relationship with the preset transformation matrix according to the first transmission parameter and/or a type of the simulated ultrasound probe in step 420 above includes steps 610 and 620 as follows.

In step 610, a plurality of channel data matrices are extracted from the echo signal of the second ultrasound wave, with the channel data matrices corresponding to different transmitting sequences of the ultrasound wave and different target scatterers, where elements in each channel data matrix are arranged according to a position sequence of the receiving array elements and an acquisition position of sampling points.

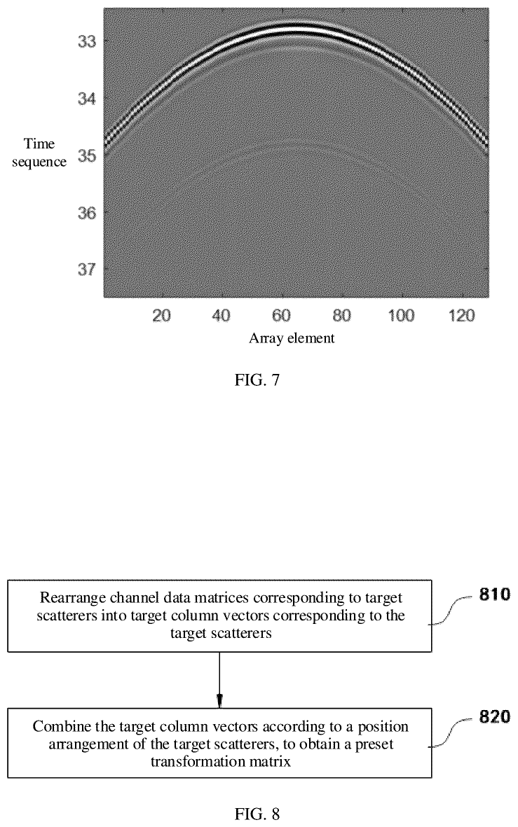

In this step, the array elements 530 on the simulated ultrasound probe 520 respectively obtain corresponding channel data, such that a plurality of channel data matrices can be obtained according to a position arrangement of the array elements 530 and a time sequence of the array elements 530 acquiring the channel data. The channel data matrix has two dimensions, which are respectively receiving array elements and sampling points. In an embodiment, a number Ne of array elements are provided on the simulated ultrasound probe 520, and each array element corresponds to a number Ns of sampling points. Therefore, there are a number Ns*Ne of channel data in one channel data matrix. Referring to FIG. 7, it is a schematic diagram of channel data in one channel data matrix. When the simulated ultrasound probe 520 is controlled to send and receive the ultrasound wave a number Nt of times, there are a corresponding number Nt of channel data matrices, where Ne, Ns, and Nt are all natural numbers, that is, one target scatterer corresponds to a number Ns*Ne*Nt of channel data in total.

In step 620, aggregation transformation is performed on the channel data matrices, to obtain the preset transformation matrix.

In this step, the channel data matrices are aggregated and transformed, and then combined into one preset transformation matrix. Referring to FIG. 8, in an embodiment, step 620 includes steps 810 and 820 as follows.

In step 810, the channel data matrices corresponding to the target scatterers are rearranged into column vectors, and the column vectors are then respectively combined into target column vectors corresponding to the target scatterers.

In this step, the channel data matrices corresponding to the target scatterers are rearranged into vectors, and the vectors are then respectively combined into target column vectors Ti corresponding to the target scatterers. If a transmitting sequence contains a number Nt of times of transmission and reception of the ultrasound wave, a number of elements of the vectorized Ti is Ns*Ne*Nt.

In step 820, the target column vectors are combined according to a position arrangement of the target scatterers, to obtain the preset transformation matrix.

In this step, Ti is the i^{th} column of the preset transformation matrix T. The target column vectors are combined according to a position arrangement of the target scatterers, to obtain the preset transformation matrix T, where the preset transformation matrix T is mathematically written as T=[T1, T2, . . . , Ti, . . . , TN]. When N target scatterers are set, a number of elements of the preset transformation matrix T is (Ns*Ne*Nt)*N.

In an embodiment, sparse processing is performed on the preset transfer matrices in the preset transformation matrix set, which saves the storage space in the memory 107.

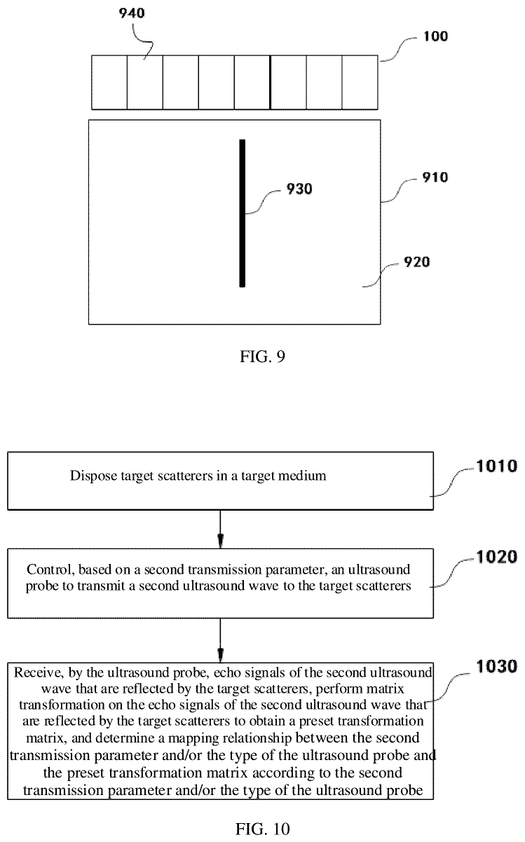

Referring to FIG. 9, in an embodiment, the preset transformation matrix is detected and obtained by means of experiments in a real-world environment. In the experimental environment, a target medium for propagating the ultrasound wave is included, where the target medium can be liquid, gas, or solid, and in an embodiment, the target medium is water 920 placed in a water tank 910. The ultrasound probe 100 is further provided in the experimental environment. Referring to FIG. 10, the preset transformation matrix is obtained through steps 1010 to 1030 as follows.

In step 1010, target scatterers are disposed in a target medium.

In an embodiment of this step, the target medium is water 920 placed in a water tank 910, and the target scatterer is a nylon rope 930 disposed in the water.

In step 1020, the ultrasound probe is controlled, according to a second transmission parameter, to transmit a second ultrasound wave to the target scatterers.

In this step, the ultrasound probe 100 sends the second ultrasound wave to the target scatterers according to the second transmission parameter.

In step 1030, echo signals of the second ultrasound wave that are reflected by the target scatterers are received by the ultrasound probe, matrix transformation is performed on the echo signals of the second ultrasound wave that are reflected by the target scatterers, to obtain the preset transformation matrix, and a mapping relationship with the preset transformation matrix is determined according to the second transmission parameter and/or a type of the ultrasound probe.

The second ultrasound wave is a real ultrasound signal. The second ultrasound wave propagates in the target medium and is reflected at the positions of the target scatterers, to generate echo signals of the second ultrasound wave that are reflected at the positions of the target scatterers. The echo signals of the second ultrasound wave are received by a plurality of array elements 940 of the ultrasound probe 100, and echo signals are then formed. Matrix transformation is performed on the echo signals of the second ultrasound wave that are reflected by the target scatterers, to obtain the preset transformation matrix, and the preset transformation matrix is added to the preset transformation matrix set. If the current preset transformation matrix is the first matrix, an empty preset transformation matrix set is created, and the current preset transformation matrix is placed into the preset transformation matrix set.

When the calculation of one preset transformation matrix is completed, the second transmission parameter and/or the type of the ultrasound probe 100 are/is changed for re-measurement, and after a new preset transformation matrix is obtained through calculation, the current preset transformation matrix is added to the preset transformation matrix set, and a relationship between the second transmission parameter and/or the ultrasound probe 100 and the current preset transformation matrix is stored. In an embodiment, the memory 107 is used to store the preset transformation matrix set, and the relationship between the second transmission parameter and/or the ultrasound probe 100 and the current preset transformation matrix.

Referring to FIG. 11, in an embodiment, performing matrix transformation on the echo signals of the second ultrasound wave that are reflected by the target scatterers, to obtain the preset transformation matrix, and determining a mapping relationship with the preset transformation matrix according to the second transmission parameter and/or a type of the simulated ultrasound probe in step 1030 above includes steps 1110 and 1120 as follows.

In step 1110, a plurality of channel data matrices are extracted from the echo signal of the second ultrasound wave, with the channel data matrices corresponding to different transmitting sequences of the ultrasound wave and different target scatterers, where elements in each channel data matrix are arranged according to a position sequence of the receiving array elements and an acquisition position of sampling points.

In this step, the array elements 940 of the ultrasound probe 100 respectively obtain corresponding channel data, such that a plurality of channel data matrices can be obtained according to a position arrangement of the array elements 940 and a time sequence of the array elements 940 acquiring the channel data. The channel data matrix has two dimensions, which are respectively receiving array elements and sampling points. In an embodiment, a number Ne of array elements are provided on the ultrasound probe 100, and each array element corresponds to a number Ns of sampling points. Therefore, there are a number Ns*Ne of channel data in one channel data matrix. When the ultrasound probe 100 is controlled to send and receive the ultrasound wave a number Nt of times, there are a corresponding number Nt of channel data matrices, where Ne, Ns, and Nt are all natural numbers, that is, one target scatterer corresponds to a number Ns*Ne*Nt of channel data in total.

In step 1120, aggregation transformation is performed on the channel data matrices, to obtain the preset transformation matrix.

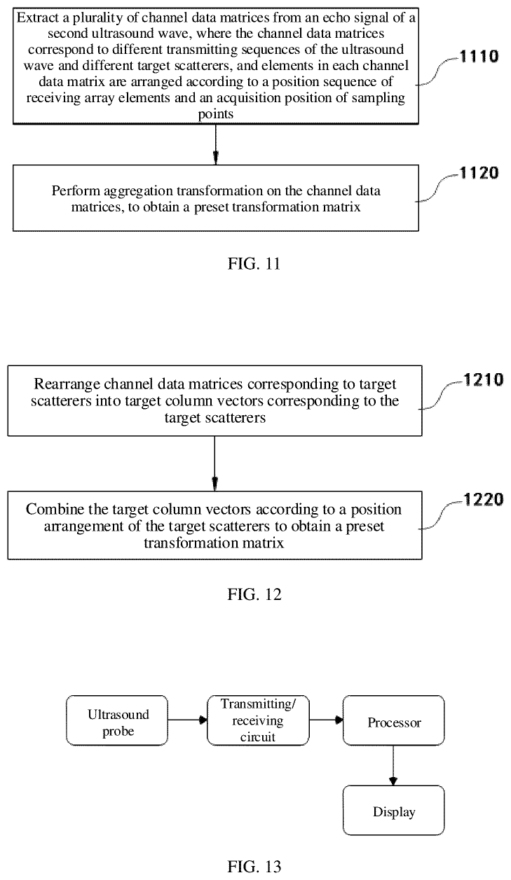

In this step, the channel data matrices are aggregated and transformed, and then combined into one preset transformation matrix. Referring to FIG. 12, in an embodiment, step 1120 includes steps 1210 and 1220 as follows.

In step 1210, the channel data matrices corresponding to the target scatterers are rearranged into column vectors, and the column vectors are then respectively combined into target column vectors corresponding to the target scatterers.

In this step, the channel data matrices corresponding to the target scatterers are rearranged into vectors, and the vectors are then respectively combined into target column vectors Ti corresponding to the target scatterers. If a transmitting sequence contains a number Nt of times of transmission and reception of the ultrasound wave, a number of elements of the vectorized Ti is Ns*Ne*Nt.

In step 1220, the target column vectors are combined according to a position arrangement of the target scatterers, to obtain the preset transformation matrix.

In this step, Ti is the i th column of the preset transformation matrix T. The target column vectors are combined according to a position arrangement of the target scatterers, to obtain the preset transformation matrix T, where the preset transformation matrix T is mathematically written as T=[T1, T2, Ti, . . . , TN]. When N target scatterers are set, a number of elements of the preset transformation matrix T is (Ns*Ne*Nt)*N.

Referring to FIG. 13, an embodiment of the disclosure provides an ultrasound imaging apparatus, the apparatus including:
an ultrasound probe;
a transmitting/receiving circuit configured to control the ultrasound probe to transmit an ultrasound wave to a target tissue and receive ultrasound echoes, to obtain an ultrasound echo signal;
a processor configured to process the ultrasound echo signal to obtain an image of the target tissue; and
a display configured to display the image of the target tissue;
the processor being further configured to perform the ultrasound imaging method in any one of the above embodiments.

Referring to FIGS. 1 and 3, an embodiment of the disclosure provides an ultrasound imaging apparatus, the apparatus including:
an ultrasound probe 100 configured to transmit a first ultrasound wave to a target tissue and receive echoes of the first ultrasound wave, the ultrasound probe including a plurality of receiving array elements, where the plurality of receiving array elements are configured to receive the echoes of the first ultrasound wave that are reflected by the target tissue, and obtain channel data respectively corresponding to the receiving array elements;
a memory 107 storing a preset transformation matrix set, where the preset transformation matrix set includes at least one preset transformation matrix;
a processor 105 configured to determine a target transformation matrix from the preset transformation matrix set according to a transmission parameter of the first ultrasound wave and/or a type of the ultrasound probe; and
a synthetic processor 104 configured to transform the plurality of channel data according to the target transformation matrix to obtain synthetic radio frequency data;
the processor 105 being further configured to perform image processing on the synthetic radio frequency data, to obtain image data of the target tissue, to obtain an image of the target tissue according to the image data, and to display the image of the target tissue.

The synthetic processor 104 includes a video memory and a graphics processing unit (GPU), the video memory being configured to cache the target transformation matrix and the plurality of channel data, and the graphics processing unit being configured to obtain the target transformation matrix and the plurality of channel data from the video memory, and transform the plurality of channel data according to the target transformation matrix, to obtain the synthetic radio frequency data.

An embodiment of the disclosure provides an electronic device, which includes a memory and a processor, where the memory stores a computer program, and when the processor executes the computer program, the ultrasound imaging method in any one of the above embodiments in the first aspect is implemented.

An embodiment of the disclosure provides a computer storage medium, which has a computer program stored thereon and is applied to an ultrasound imaging device, where when the computer program is executed by a processor, the ultrasound imaging method in any one of the above embodiments in the first aspect is implemented.

An embodiment of the disclosure provides a computer program product or computer program including computer instructions, where the computer instructions are stored in a computer-readable storage medium. A processor of a computer device reads the computer instructions from the computer-readable storage medium, and the processor executes the computer instructions, so that the computer device performs the ultrasound imaging method in any one of the above embodiments in the first aspect.

In several embodiments provided in the disclosure, it should be understood that the disclosed systems, apparatuses, and methods can be implemented in other ways. For example, the device embodiments described above are merely exemplary. For example, the division of units is only a logic function division. In actual implementation, there may be other division methods, for example, a plurality of units or assemblies may be combined or integrated into another system, or some features may be omitted or not implemented. In addition, the mutual coupling or direct coupling or communication connection shown or discussed may be indirect coupling or communication connection through some interfaces, apparatuses, or units, and may be in electrical, mechanical, or other forms.

The units described as separate parts may or may not be physically separated, and the parts displayed as units may or may not be physical units, that is, may be located in one place, or may be distributed over a plurality of network units. Some or all of the units can be selected according to actual needs to achieve the objectives of solutions of the embodiments.

Additionally, the functional units in the embodiments of the disclosure may be integrated into one processing unit or may also exist as being physically separate, or two or more of the units may be integrated into one unit. The integrated units described above may be implemented in the form of hardware or software function units.

If the integrated unit is implemented in the form of the software function units and sold or used as an independent product, it may be stored in a computer-readable storage medium. According to such an understanding, the technical solutions of the disclosure essentially, or the part contributing to the prior art may be implemented in the form of a software product. The computer software product may be stored in a storage medium and includes several instructions for instructing a computer apparatus (which may be a personal computer, a server, or a network apparatus) to perform all or some of the steps of the methods in the embodiments of the disclosure. The foregoing storage medium includes: a U disk, a removable hard disk, a read-only memory (ROM for short), a random access memory (RAM for short), a magnetic disk or an optical disk, and other media that can store program codes.

It should also be understood that various implementations provided in the embodiments of the disclosure may be combined in any manner, to achieve different technical effects.

The embodiments of the disclosure have been described above, but the disclosure is not limited to the above-mentioned embodiments. Those skilled in the art can also make various equivalent variations or replacements without departing from the shared conditions of the spirit of the disclosure. All of these equivalent variations or replacements shall be included in the scope of the disclosure as defined in the claims.

What is claimed is:

1. An ultrasound imaging method, comprising:
transmitting a first ultrasound wave to a target tissue via an ultrasound probe;
receiving, via the ultrasound probe, echo of the first ultrasound wave that is reflected by the target tissue to obtain an echo channel signal, wherein the echo channel signal comprises a plurality of channel data corresponding to a plurality of receiving array elements of the ultrasound probe;
determining a target transformation matrix from a transformation matrix set according to a transmission parameter of the first ultrasound wave and/or a type of the ultrasound probe;
transforming the plurality of channel data according to the target transformation matrix to obtain synthetic radio frequency data;
performing image processing on the synthetic radio frequency data to obtain image data of the target tissue; and
obtaining an image of the target tissue according to the image data, and displaying the image of the target tissue.

2. The ultrasound imaging method of claim 1, wherein the plurality of channel data in the echo channel signal, the target transformation matrix, and the synthetic radio frequency data constitute a linear forward model, and transforming the plurality of channel data according to the target transformation matrix to obtain synthetic radio frequency data comprises:
solving an inverse problem for the linear forward model according to the target transformation matrix and the plurality of channel data in the echo channel signal to obtain the synthetic radio frequency data.

3. The ultrasound imaging method of claim 1, wherein transforming the plurality of channel data according to the target transformation matrix to obtain synthetic radio frequency data comprises:
rearranging the plurality of channel data into column vectors according to a channel order of the receiving array elements of the ultrasound probe to obtain the plurality of channel data in a column vector form; and
transforming the plurality of channel data in the column vector form according to the target transformation matrix to obtain the synthetic radio frequency data in a column vector form.

4. The ultrasound imaging method of claim 2, wherein the linear forward model is expressed as:
Y=TX,
wherein Y is the echo channel signal, X is the synthetic radio frequency data, T is the target transformation matrix, Y and X are both data in a column vector form, a number of elements of Y is Ns*Ne*Nt, T is an N1*N2 matrix, N1=Ns*Ne*Nt, N2 is a number of beamforming points, Ns is a number of sampling points of the first ultrasound wave, Ne is a number of channels, and Nt is a number of times the ultrasound probe transmits ultrasound waves; and
solving an inverse problem for the linear forward model comprises solving an inverse problem for X given that Y and T are known in the linear forward model.

5. The ultrasound imaging method of claim 2, wherein solving an inverse problem for the linear forward model comprises:
performing matrix inversion on the target transformation matrix to obtain a target inverse transformation matrix, and performing matrix multiplication on the target inverse transformation matrix and the plurality of channel data to obtain the synthetic radio frequency data; or
performing least-squares transformation on the target transformation matrix and the plurality of channel data to solve for an inverse matrix to obtain the synthetic radio frequency data; or
performing solving by means of a related regularization method for the target transformation matrix and the plurality of channel data to obtain the synthetic radio frequency data; or
performing solving by means of compressed sensing for the target transformation matrix and the plurality of channel data to obtain the synthetic radio frequency data; or
inputting the target transformation matrix and the plurality of channel data into a pre-trained deep learning model for inverse problem solving to obtain the synthetic radio frequency data.

6. The ultrasound imaging method of claim 2, wherein when solving the inverse problem for the linear forward model, the plurality of channel data and the target transformation matrix are sent to a video memory of an ultrasound imaging apparatus, and a graphics processing unit (GPU) of the ultrasound imaging apparatus is used to solve the inverse problem to obtain the synthetic radio frequency data.

7. The ultrasound imaging method of claim 1, wherein the ultrasound probe is a linear array probe, a convex array probe or a phased array probe.

8. The ultrasound imaging method of claim 1, wherein the transmission parameter comprises a focused wave transmission parameter, a plane wave transmission parameter, or a diverging wave transmission parameter.

9. The ultrasound imaging method of claim 1, wherein, the transformation matrix set comprises a plurality of transfer matrices, and each of the transfer matrices is obtained by:
configuring an ultrasound detection simulation model, wherein the ultrasound detection simulation model comprises a simulated ultrasound probe, a simulation environment, and simulated target scatterers disposed in the simulation environment; and
controlling, according to a first transmission parameter, the simulated ultrasound probe to transmit a second ultrasound wave to the target scatterers, receiving, by the simulated ultrasound probe, echo signals of the second ultrasound wave that are reflected by the target scatterers, performing matrix transformation on the echo signals of the second ultrasound wave that are reflected by the target scatterers to obtain the transformation matrix, and determining a mapping relationship between the first transmission parameter and/or a type of the simulated ultrasound probe and the transformation matrix according to the first transmission parameter and/or the type of the simulated ultrasound probe.

10. The ultrasound imaging method of claim 1, wherein, the transformation matrix set comprises a plurality of transfer matrices, and each of the transfer matrices is obtained by:
disposing target scatterers in a target medium;
controlling, according to a second transmission parameter, the ultrasound probe to transmit a second ultrasound wave to the target scatterers; and
receiving, by the ultrasound probe, echo signals of the second ultrasound wave that are reflected by the target scatterers, performing matrix transformation on the echo signals of the second ultrasound wave that are reflected by the target scatterers to obtain the transformation matrix, and determining a mapping relationship between the second transmission parameter and/or the type of the ultrasound probe and the transformation matrix according to the second transmission parameter and/or the type of the ultrasound probe.

11. The ultrasound imaging method of claim 10, wherein the target medium is water contained in a water tank, and the target scatterer is a nylon rope disposed in the water in the water tank.

12. The ultrasound imaging method of claim 9, wherein performing matrix transformation on the echo signals of the second ultrasound wave that are reflected by the target scatterers to obtain the transformation matrix comprises:
extracting a plurality of channel data matrices from the echo signals of the second ultrasound wave, wherein, the channel data matrices correspond to different transmitting sequences of the second ultrasound wave and different target scatterers, and elements in each of the channel data matrices are arranged according to a position sequence of the receiving array elements and an acquisition position of sampling points; and
performing aggregation transformation on the channel data matrices to obtain the transformation matrix.

13. The ultrasound imaging method of claim 12, wherein performing aggregation transformation on the channel data matrices to obtain the transformation matrix comprises:
rearranging the channel data matrices corresponding to the target scatterers into target column vectors corresponding to the target scatterers; and
combining the target column vectors according to a position arrangement of the target scatterers to obtain the transformation matrix.

14. The ultrasound imaging method of claim 9, further comprising:
performing sparse processing on the transfer matrices in the transformation matrix set.

15. An ultrasound imaging apparatus, comprising:
an ultrasound probe;
a transmitting/receiving circuit configured to control the ultrasound probe to transmit an ultrasound wave to a target tissue and receive ultrasound echoes to obtain an echo channel signal, wherein the echo channel signal comprises a plurality of channel data corresponding to a plurality of receiving array elements of the ultrasound probe;
a processor configured to
determining a target transformation matrix from a transformation matrix set according to a transmission parameter of the ultrasound wave and/or a type of the ultrasound probe;
transforming the plurality of channel data according to the target transformation matrix to obtain synthetic radio frequency data;
performing image processing on the synthetic radio frequency data to obtain image data of the target tissue; and
obtaining an image of the target tissue according to the image data; and
a display configured to display the image of the target tissue.

16. An ultrasound imaging apparatus, comprising:
an ultrasound probe configured to transmit a first ultrasound wave to a target tissue and receive echoes of the first ultrasound wave, the ultrasound probe comprising a plurality of receiving array elements, wherein the plurality of receiving array elements are configured to receive the echoes of the first ultrasound wave that are reflected by the target tissue to obtain channel data respectively corresponding to the receiving array elements;
a memory storing a transformation matrix set, wherein the transformation matrix set comprises at least one transformation matrix;
a processor configured to determine a target transformation matrix from the transformation matrix set according to a transmission parameter of the first ultrasound wave and/or a type of the ultrasound probe; and
a synthetic processor configured to transform the channel data according to the target transformation matrix to obtain synthetic radio frequency data;
wherein the processor being further configured to perform image processing on the synthetic radio frequency data to obtain image data of the target tissue, to obtain an image of the target tissue according to the image data, and to display the image of the target tissue.

17. The ultrasound imaging apparatus of claim 16, wherein the synthetic processor comprises a video memory and a graphics processing unit, the video memory being configured to cache the target transformation matrix and the channel data, and the graphics processing unit being configured to obtain the target transformation matrix and the channel data from the video memory and transform the channel data according to the target transformation matrix to obtain the synthetic radio frequency data.
